(19) United States
(12) Patent Application Publication
(10) Pub. No.: US 2023/0233792 A1
(43) Pub. Date: Jul. 27, 2023

(54) VENTILATION SYSTEM WITH IMPROVED VALVING

(71) Applicant: Ryan Redford, Irvine, CA (US)

(72) Inventor: Ryan Redford, Irvine, CA (US)

(21) Appl. No.: 18/128,859

(22) Filed: Mar. 30, 2023

Related U.S. Application Data
(63) Continuation of application No. 18/023,288, filed on Jan. 1, 0001.
(60) Provisional application No. 63/240,298, filed on Sep. 2, 2021.

(51) Int. Cl. A61M 16/20

(52) U.S. Cl. CPC A61M 16/203

(57) ABSTRACT

A respiratory ventilators system having an inlet configured to be connected to a pressurized air or gas source; an outlet configured to be connected to a patient interface; a valve in-line between the inlet and the outlet; and a control unit configured to control the valve for controlling flow of pressurized air or gas from the source to the patient, wherein the valve includes an air or gas reservoir or accumulator incorporated into the valve body.

### CROSS REFERENCE TO RELATED APPLICATIONS

This application is a continuation of U.S. patent application Ser. No. 18/023,288 filed Feb. 24, 2023, which is a national stage application of PCT/US2022/042531 filed Sep. 2, 2022, which claims benefit to U.S. Provisional Patent Application Ser. No. 63/240,298, filed Sep. 2, 2021, the contents of which are incorporated herein by reference.

### BACKGROUND OF THE DISCLOSURE

The present disclosure relates generally to respiratory care systems, and more particularly, to mechanical ventilation systems or respiratory care systems, i.e., ventilators or respirators. The disclosure has particular utility for supplying respiratory support for a human or animal patient whose breathing is compromised by disease and will be described in connection with such utility, but also may be utilized for treating patients suffering from sleep apnea or for use as a component of an anesthesia system.

The current Covid-19 pandemic has highlighted the need for mechanical ventilation systems for respiratory compromised patients. Respiratory treatment apparatus can function to supply a patient with a supply of clean breathable gas (usually air, with or without supplemental oxygen) at a therapeutic pressure or pressures, at appropriate times during the subject's breathing cycle. Therapeutic pressure assist may be implemented in a synchronized fashion with the patient's breathing so as to permit greater pressures during a patient's normal breathing inspiration cycle and lower pressures during expiration. Therapeutic pressure assist also may be implemented to override a patient's normal breathing inspiration cycle.

Respiratory care systems typically include a gas or air flow generator or source of compressed gas or air, an air filter, a nasal, oral or full face mask, an air delivery conduit connecting the flow generator to the mask, various sensors and a microprocessor-based controller. Optionally, in lieu of a mask, a tracheotomy tube may also serve as a patient interface. The flow generator may include a servo-controlled motor and an impeller that forms a blower. In some cases a brake for the blower motor may be implemented to more rapidly reduce the speed of the blower so as to overcome the inertia of the motor and impeller. The braking can permit the blower to more rapidly achieve a lower pressure condition in time for synchronization with the patient's expiration despite the inertia. In some cases the flow generator also may include a valve capable configured to discharge generated air to atmosphere as a means for altering the pressure delivered to the patient as an alternative to motor speed control. The sensors measure, amongst other things, motor speed, mass flow rate and outlet pressure, such as with a pressure transducer or the like. The apparatus optionally may include a humidifier and/or heater elements in the path of the air delivery circuit. The controller may include data storage capacity with or without integrated data retrieval and display functions.

Respiratory care systems may be used for the treatment of many conditions, for example respiratory insufficiency or failure due to lung, neuromuscular or musculoskeletal disease and diseases of respiratory control. They may also be used for conditions related to sleep disordered breathing (SDB) (including mild obstructive sleep apnea (OSA)), allergy induced upper airway obstruction or early viral infection of the upper airway.

The current Covid-19 pandemic has stretched the current supply of respiratory care systems. Hospitals have been forced to share respiratory care systems, i.e., ventilators between two patients. Hospitals also have resorted to adapting apparatus conventionally used for obstructive sleep apnea as a poor substitute for conventional ventilators.

Also, current ventilators are complex it expensive devices which require constant supervision and adjustment, and which are prone to a breakdown.

### SUMMARY OF THE DISCLOSURE

The present a disclosure provides a simple low cost ventilator which overcomes the aforesaid and other disadvantages of the current state of the art ventilators.

More particularly, the present disclosure provides a ventilator having a significant advantages over current ventilators in terms of cost, size reduction, weight reduction, power reduction, noise reduction and reliability. One key to the instant ventilator of the present disclosure is a unique air or gas flow valve having an air or gas reservoir or accumulator incorporated into the valve. Incorporating an air or gas reservoir or accumulator into the value simplifies the construction and cost of the system, while providing improved response time thereby providing better patient support. Conventional ventilators employ proportional solenoid valves (PSOL valves) or turbine-based designs, where the core flow/pressure regulating component is a high-cost, multi-part item (order $1,500-$2,000). Also, in practice, static friction on the guide posts of the plunger of conventional PSOL valves may impair sensitivity of the valve, which in turn may result in hysteresis effects. To overcome the above and other disadvantages of conventional ventilators, the instant disclosure employs a novel low cost air or gas valve which has an integral air or gas reservoir or accumulator incorporated into the valve and which valve essentially consists of five primary elements and essentially one moving part.

In one embodiment, respiratory ventilator system of the present disclosure comprises an inlet configured to be connected to a pressurized air or gas source; an outlet configured to be connected to a patient interface; a valve in-line between the inlet and the outlet; and a control unit configured to control the valve for controlling flow of pressurized air or gas from the source to the patient, wherein the valve includes an air or gas reservoir or accumulator incorporated into the valve body.

In one preferred embodiment the valve comprises a valve gate controlled by a linear drive mechanism, preferably a servomechanism, a mechanical screw drive or a voice coil drive.

The patient interface may be selected from the group consisting of a mask, an intubation tube and a tracheotomy cannula, and pressurized air or gas source may be selected from the group consisting of an air canister, a compressor, an air pump, and pressurized airline.

The present disclosure also provides a method for assisting breathing of a patient in need of same, comprising: providing a ventilation system as above described; connecting the ventilation system to a source of pressurized air and to a patient interface; initiating a flow of air or gas to the ventilator system to precharge the air or gas reservoir or accumulator and controlling the flow of gas through the ventilation system by opening and closing the valve.

In another embodiment of the disclosure, the ventilator system includes a heater and/or a humidifier for conditioning the air or gas.

The valve may be opened and closed in response to the patient's normal breathing cycle, or the valve may be opened and closed to introduce a flow of air or gas to override the patient's normal breathing cycle.

The patient may be a human animal; or a non-human animal.

### BRIEF DESCRIPTION OF THE DRAWINGS

Further features and advantages of the instant disclosure will be seen from the following description, taken in conjunction with the accompanying drawings, wherein like numerals depict like parts, and wherein:

FIG. 1 is a schematic diagram of a ventilator system incorporating compact ventilation device shown connected to a patient in accordance with the present disclosure;

FIG. 2 is a perspective view of a compact ventilation device made in accordance with the present disclosure;

FIG. 3 is cross-sectional view of a functional element diagram of the valve component of the compact ventilation device in accordance with a preferred embodiment of the instant disclosure;

FIGS. 4 and 5 are cross-sectional functional element diagrams of the valve component of the compact ventilation device in accordance with the present disclosure;

FIG. 6 is a diagram showing force and moment balance of the valve component of the subject disclosure;

FIG. 7 is an exploded view of the valve component in accordance with the present disclosure;

FIG. 8 is a flow diagram illustrating operation by the compact ventilation device of the subject disclosure; and

FIGS. 9A-9C are graphs illustrating triggered airflow into in accordance with the present disclosure.

### DETAILED DESCRIPTION OF THE PRESENT INVENTION

In the following detailed description the terms “air” and “gas”, and the terms “respirator” and “ventilator”, respectively, are used interchangeably.

The present respiratory treatment apparatus of the present disclosure provides supplemental air or oxygen to a patient in intermittent time intervals, based either on the patient's natural tidal breathing cycle, or based on a programmed a breathing cycle.

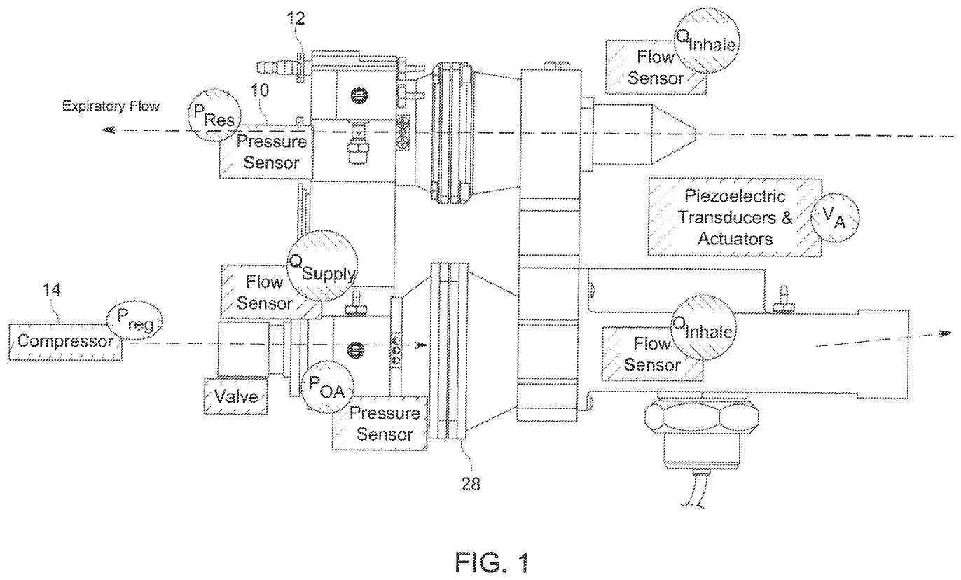

Referring to FIG. 1, a respiratory ventilator system 10 includes a ventilation controller 12 connected to a pressurized gas source 14. The pressurized gas source could be a pressurized air or air/oxygen gas canister, a compressor or air pump as illustrated, or a pressurized airline. The ventilation controller 12 which will be described below in detail, permits the flow of pressurized gas to the patient through a gas supply line at 16 which is fixed to a patient interface such as a nasal or full face mask 18 worn by a patient 22. Alternatively, the patient interface 18 may comprise an intubation tube or tracheotomy cannula. Completing the system is a capnography monitor 24 of which senses and measures inhalation and/or exhalation airflow from the patient, and a command input and monitor 26. The capnography monitor 24 and the command input and monitor 26 are conventional and need not be further described for an understanding of the instant disclosure.

Central to the ventilator system 10 of the current disclosure is a gas or airflow control valve 28 having an integral gas or air reservoir or accumulator as will be described below.

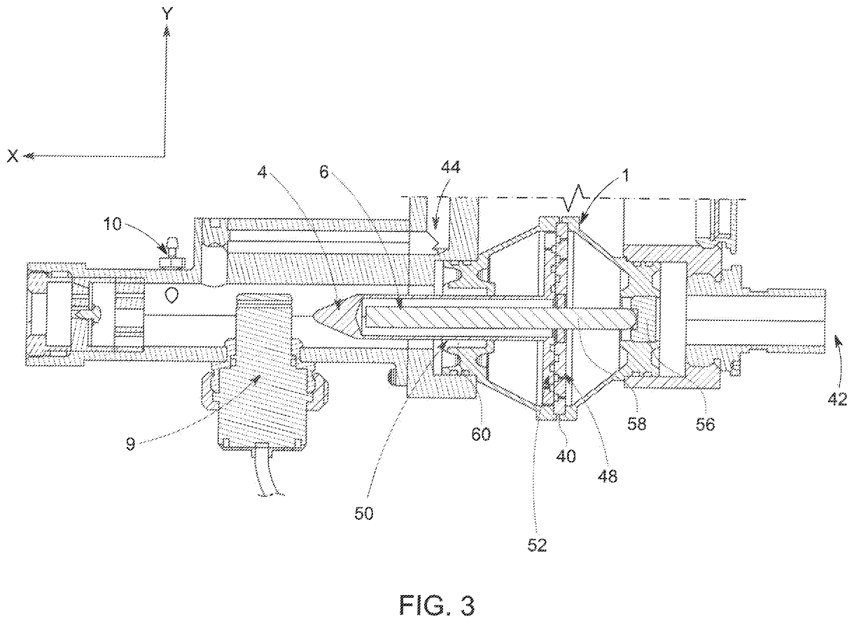

Referring now to FIGS. 3-5, the gas or airflow control valve 28 includes a valve housing 40 which contains the active elements of the gas or airflow control valve 28. The gas supply inlet 42 is shown on the negative X axis face and the gas source outlet 44 is on the positive X face. Additionally, the housing 40 creates a gas reservoir or accumulator 46. The gas supply inlet 42 can interface with a standard hospital O₂ source or with any gas source, e.g., a gas canister or compressor.

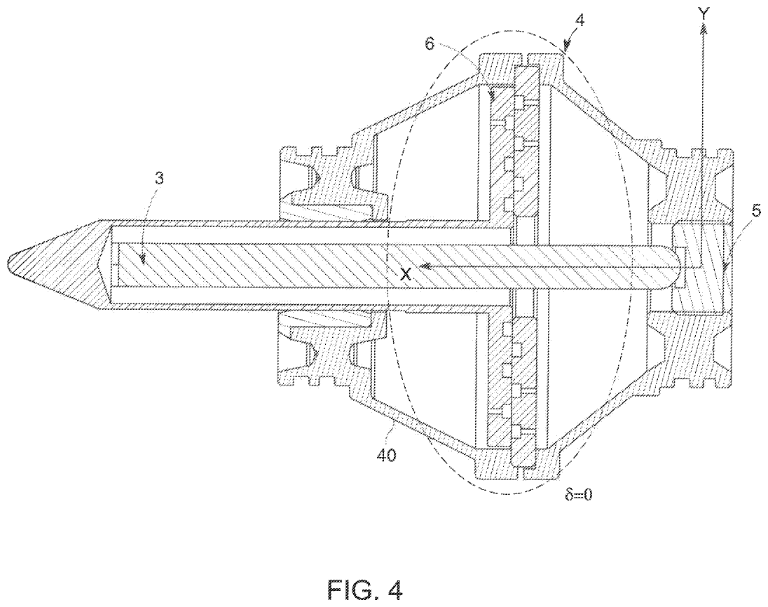

A valve gate 48 described below with reference to FIGS. 3 and 4 controls the source flow rate, QSource(t) based on its position along the X axis. The face on the negative Z surface slides along the X axis on the valve housing slide surface 50. The distance between the valve gate face on the positive X axis of the Gate and the Valve Housing Seal Surface 52, 6, determines the flow resistance by creating a resistance channel between the valve housing seal surface and the valve gate Y-Z face on the positive X axis.

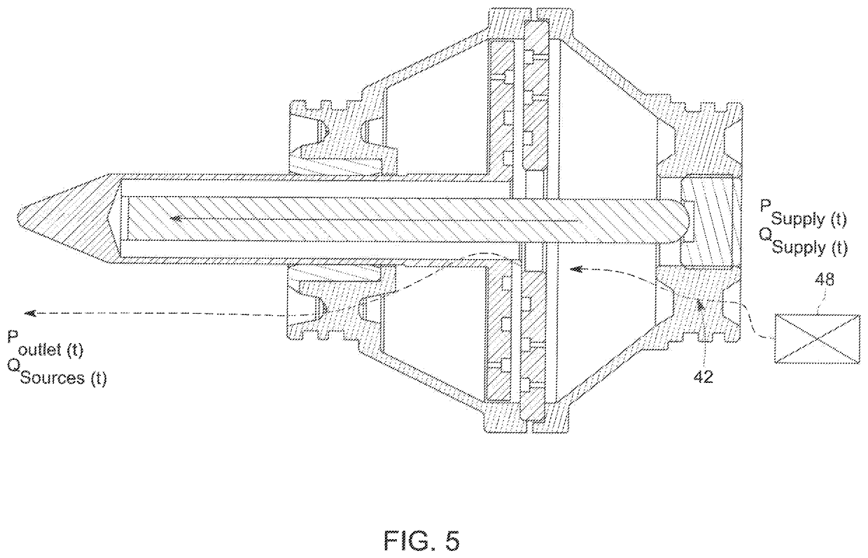

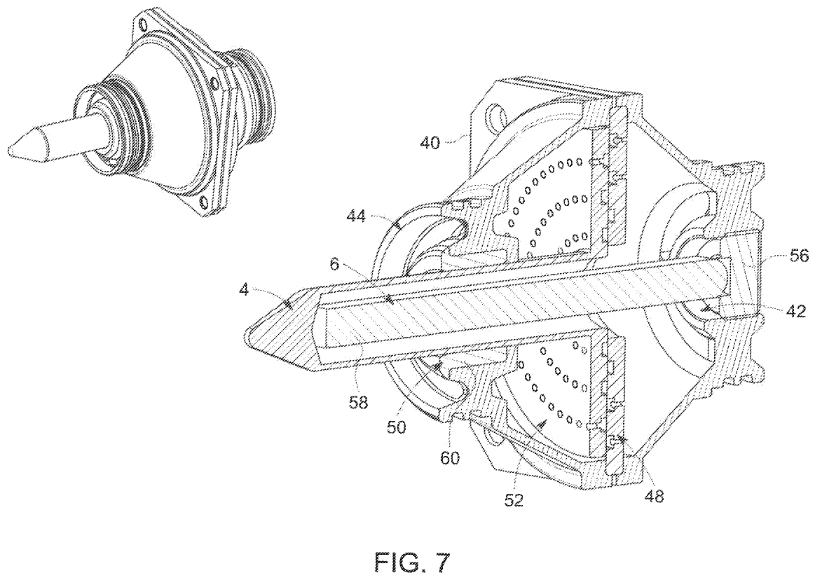

Referring in particular to FIGS. 5 and 7, the gas or airflow control valve 28 includes a valve gate 48 configured to slide along the X axis of a valve slide surface 50 setting its position along the X axis. The gas or airflow control valve 28 also includes a linear actuator 54 such as a servo mechanism formed of an electro-strictive material such as PZT or PMN, a magneto-strictive material, or a mechanical screw drive of a voice coil drive or other linear drive mechanism. It's length and the resulting gate valve position is controlled under closed loop control based on the desired source flow rate, Q_{Source}(t) or flow source pressure, P_{Source}(t). The valve gate 48 also could be driven under open loop control.

A preload force in the negative X direction is applied to the valve gate 48 assembly by a spring assembly 56.

A set screw 50 drives the valve gate 48 in the X direction, setting both a spring assembly preload force and the initial position of the valve gate 48 along the X axis.

A spring plunger 58 provides a preload to the valve gate 48 in the negative Z direction. The intent is to continually maintain a gas-tight seal between the valve gate 48 and valve housing slide surface 50.

A gasket 60 maintains a gas-tight seal between the X-Z surfaces of the valve housing and the valve gate 48.

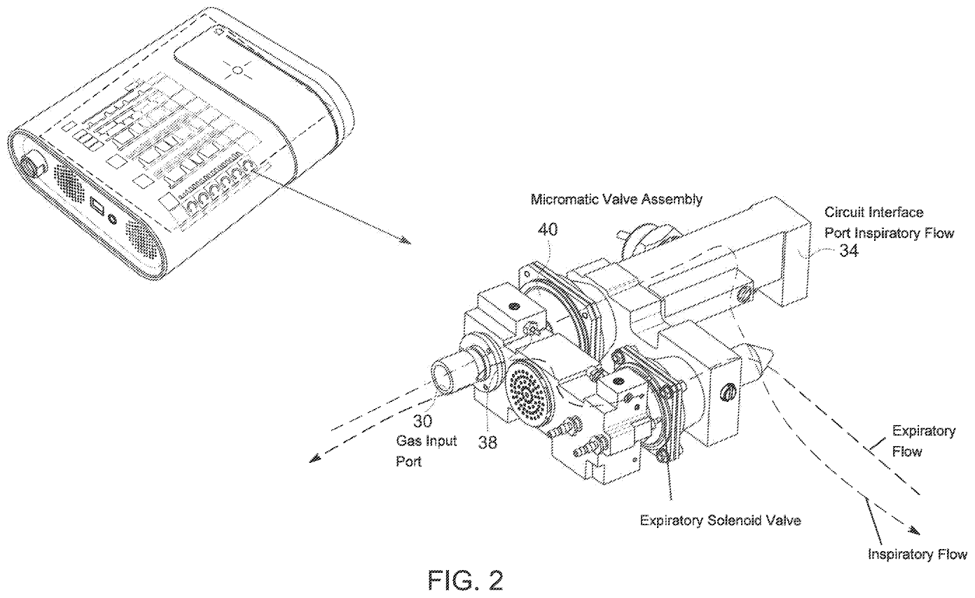

Referring again to FIG. 2, the ventilation controller 12 includes an air or gas input port 30 which is connected to the gas or airflow control valve 28. Control valve 28 has an outlet 32 that is connected to a port that includes an inspiratory flow connection 34 and an expiratory flow port 36 which in turn is connected to an expiratory flow valve 38.

Expiratory flow valve 38 may be vented to atmosphere, or connected to scrub CO₂ and recycle through gas input port 30. The system also include expiratory flow sensors or breathing sensors 40 for sensing the patient's breathing, and connections from the sensors for triggering the valve 28. The sensors may comprise air flow sensors, temperature sensors, sound sensors, CO₂ sensors or motion or strain sensors for detecting movement of a patient's chest.

A valve cover 62 encloses the X-Z face of the valve housing, one on the positive Y axis and one on the negative Y axis. These covers create a gas-tight seal between the valve housing 40 and the atmosphere.

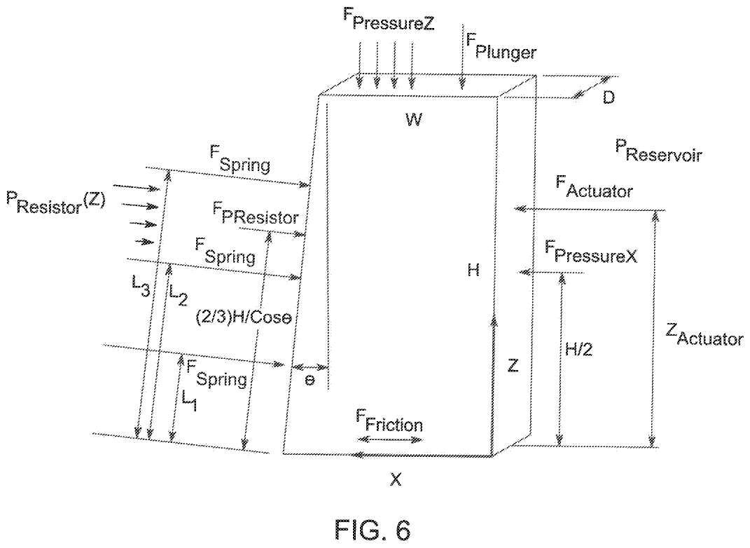

Referring again to FIGS. 4-6, on the left of FIG. 4 shows the valve in a closed position, δ=0, and the valve flow resistance, R_{Valve}(0) infinite. FIG. 4 shows the valve assembly with the gate moved a distance δ, in the negative direction along the X axis. As a result the valve resistance is no longer infinite and gas flows from the reservoir to the Gas Source outlet as shown.

The valve flow resistance, R_{Valve}(δ) is calculated as follows:

Source flow, Q_{Source}(t), is governed by Equation 3 where:

### Reservoir Pressure, P_{Reservoir}(t)

### Outlet Pressure, P_{Outlet}(t)

Source flow rate, Q_{Source}(t)

### Valve Height, H_{Valve}

Valve Depth along the Y axis, D_{Valve}
Valve Gate distance from valve housing sealing surface, δ

$${A}_{Resistance} ( δ ) = Resistance Channel Cross - sectional area \qquad Equation 1 )$$

$$= {D}_{Valve} δ$$

Gas Dynamic Viscosity, η (mass/(distance−time)

$${R}_{Valve} ( δ ) = Vent flow resistance through the resistance channel \qquad Equation 2 )$$

$$= ( 8 η / π ) {H}_{Valve} / {{A}_{Resistance} ( δ )}^{2}$$

Source Flow Rate can then be determined by the following relationship:

Q_{Source}(t)=(P_{Reservoir}(t)−P_{Outlet}(t))/R_{Valve}(b) Equation 3)

The gas reservoir region in the valve housing is required, for while the average Source flow rate, Q_{Source}(t) does not exceed the available supply flow rate, Q_{Supply}(t), but the peak flow rate for Q_{Source}(t) does. This difference is made up from gas stored in the reservoir.

The force and moment balance for a generalized Valve Gate is illustrated in FIG. 5 Governing equations for both the force balance and moment balance are provided in the Equations 4-12.

$${P}_{Reservoir} = Reservoir pressure$$

$$θ = Valve Gate Angle$$

$$W = Valve Gate Width$$

$$H = Valve Gate Height$$

$${H}_{Valve} = H / Cos θ \qquad Equation 4 )$$

$${D}_{Valve} = Valve Gate Depth$$

$${L}_{1} , {L}_{2} \& {L}_{3} = Spring Distance$$

$${c}_{Friction} = Wedge Friction Coefficient$$

$${Z}_{Actuator} = Actuator Distance from Z = 0$$

$${P}_{Reservoir} = Reservoir gas pressure$$

$${F}_{PressureX} = force from chamber pressure in - X direction \qquad Equation 5 )$$

$$= - {P}_{Reservoir} HD$$

$${F}_{PressureZ} = force from chamber pressure in - Z direction \qquad Equation 6 )$$

$$= - {P}_{Reservoir} ( WD + H tan θ D / 2 )$$

$${F}_{Plunger} = Force from Spring Plunger in Z direction$$

$${P}_{Resistor} ( Z ) = Pressure along flow resistor wall \qquad Equation 7 )$$

$$\approx {P}_{Reservoir} ( Z / H )$$

$${F}_{PResistor} = Force on Resistor wall from Pressure due to flow \qquad Equation 8 )$$

$$= ( {P}_{Reservoir} / 2 ) {D}_{Valve} {H}_{Valve}$$

$${F}_{Spring} = Force provided by preload spring$$

$${F}_{Actuator} = Force provided by positioning actuator$$

$${F}_{Z} = {F}_{Plunger} + {F}_{PressureZ} + 3 {F}_{Spring} Sin θ \qquad Equation 9 )$$

$${F}_{Friction} = Friction force in X direction \qquad Equation 10 )$$

$$= {F}_{Z} {c}_{Friction}$$

### X Force Balance

(3F_{Spring}+F_{PResistor})Cos θ=F_{PressureX}+F_{Actuator} Equation 11)

### Moment Balance About Y Axis

F_{Spring}(L₁+L₂+L₃)Cos θ²+F_{PResistor}((2/3)H/COS θ)Cos θ²=F_{PressureX}H/2+F_{Actuator}Z_{Actuator} Equation 12)

Referring also to FIG. 7, the valve assembly 28 controls source gas flow by varying flow resistance, R_{Valve}(δ). This is accomplished by changing the actuator 54 length, ΔX, which in turn results in moving the valve gate 48 along the X axis in corresponding gap between the valve gate 48 and valve housing seal surface 50 by δ. Reservoir pressure, P_{Reservoir}(t) is monitored and utilized by a pressure sensor 70 to calculate the required ΔX command that controls Q_{Source}(t) as outlined by Equation 3.

Gas flows through a flow rate sensor in line in the gas supply inlet 42 measuring source flow, Q_{Source}(t) that is a function of time, t. This flow measurement is utilized by the gas source controller & sensor/user interface to calculate the required ΔX command that controls Q_{Source}(t) as outlined by Equation 3.

As in the case of conventional ventilators, inlet gas or flow may require humidification and or heating. This is accomplished by commands from the controller to a humidification and heat module 72, which communicates with the reservoir 46, which adds water vapor, adding humidity to the gas flow, by either heating and subsequent evaporation of water, piezo atomization of water or other conventional methods of adding water to the gas flow. The gas can also be heated by this module as the gas flows through.

Gas flows through a relative humidity sensor measuring gas relative humidity, RH(t) that is a function of time, t. This measurement is utilized by the controller to generate the desired RH command, RH_{Command}(t) as a function of time.

A temperature and pressure source module measures gas temperature, T(t). This temperature measurement is utilized by the controller and sensor/user Interface to calculate the heating command, T_{Command}(t), to the Humidification and Heat Module to control gas temperature.

The temperature and pressure source module also may measure gas outlet pressure, P_{Outlet}(t). This pressure is utilized by the controller and sensor/user interface to calculate the required ΔX command that controls Q_{Source}(t) as outlined by Equation 3. The outlet of the temperature and pressure Module interfaces with a gas supply line that terminates with a pressurized nasal ventilator or other patient respiratory device such as a mask, cannula or intubation tube.

The gas source controller and sensor/user interface includes a sensor interface required for controlling the gas source flow rate, Q_{Source}(t), pressure, P_{Outlet}(t), temperature T(t) and relative humidity, RH(t). It generates the actuator command, ΔX(t), the temperature command T_{Command}(t) and the relative humidity command RH_{Command}(t). It also interfaces with the User Command Input Device & Status Monitor, receiving the user defined command set for gas source flow rate, Q_{Source}(t), pressure, P_{Outlet}(t), T(t) and RH(t). The gas source controller and sensor/user interface also provides sensor readings to the user command input device and status monitor.

The user command input device and status monitor allows the user to generate commands for gas source flow rate, Q_{Source}(t), pressure, P_{Outlet}(t), T(t) and RH(t). It also displays sensor readings. This device can be an I-Pad-like interface that communicates with the pressurized nasal ventilator assembly in a wired or wireless fashion.

The gas supply line can be a standard O₂ line. The gas supply line also can be insulated in order to minimize gas heat loss when traveling from the gas source to the pressurized nasal ventilator assembly. The gas supply line also can incorporate an electrical heating element in order to maintain gas temperature, and also can incorporate a power and data wire set to provide power to the pressurized nasal ventilator assembly and receive sensor data from the pressurized nasal ventilator assembly. Since, the gas supply line has a know flow resistance, R_{GSL}, the pressure at the point of entry to the pressurized nasal ventilator Gas Port, P_{Source}(t) can be calculated as a result of knowing Q_{Source}(t), P_{Outlet}(t) and R_{GSL} by the equation P_{Source}(t)=P_{Outlet}(t)−Q_{Source}(t) R_{GSL}.

Additional sensors can provide input for controlling the gas source assembly. These include but are not limited to air chamber pressure, P_{Chamber}(t), air chamber temperature, T_{AC}, air chamber relative humidity, RH_{AC}, ETCO₂ and or O₂ measurements sampled from the pressurized nasal ventilator assembly air chamber, impedance-based devices that monitor respiratory rate and tidal volume through chest cavity motion such as systems.

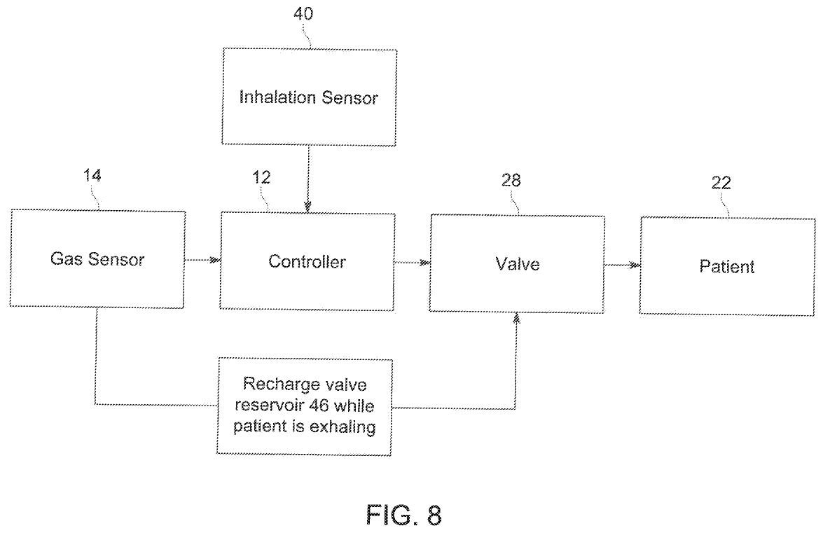

Referring to FIG. 8, overall operation is as follows: a gas source 14 supplies pressurized gas to the ventilation controller 12 which opens valve 28 to supply of gas to the patient 22 at the required frequency, flow rate and pressure to support a patient's breathing. Due to the presence of a supply of pressurized gas or air in the air or gas reservoir 46 incorporated into the valve 28, the delivery of pressurized air or gas to the patient 22 proceeds essentially instantaneously with the opening of the valve. The air or gas reservoir 48 is recharged while the patient is exhaling.

The resulting ventilator system of the present disclosure is a low cost, relatively simple device, compared to conventional ventilation devices, that is robust, and conveniently small and light weight, and exceptionally fast in responding to patient needs.

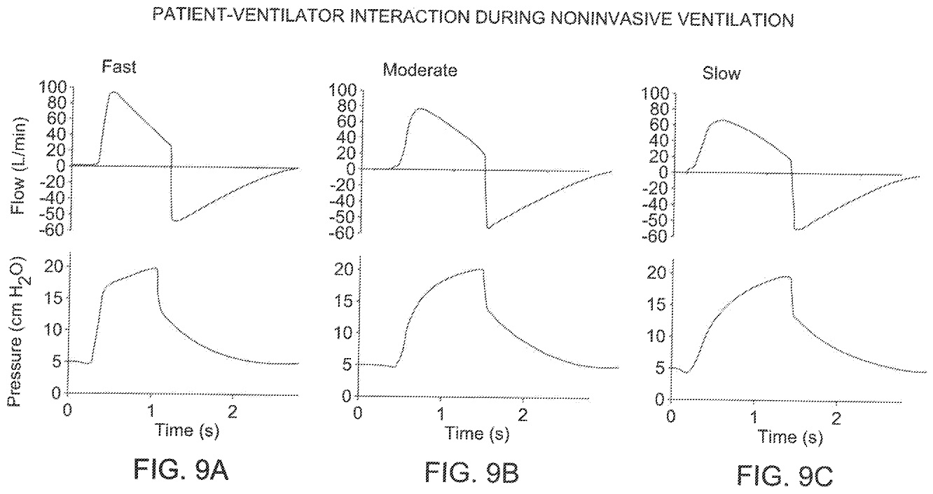

FIGS. 9A-9C are flow and pressure wave forms illustrating 3 rise times (pressure support) for a patient.

1. A respiratory ventilator system comprising:
an inlet configured to be connected to a pressurized air or gas source;
an outlet configured to be connected to a patient interface;
a valve in-line between the inlet and the outlet; and
a control unit configured to control the valve for controlling flow of pressurized air or gas from the source to a patient,
wherein the valve wherein the valve comprises a valve gate controlled by a linear drive servomechanism, and includes an air or gas reservoir or accumulator incorporated into a body of the valve.

2. The respiratory ventilator system of claim 1, with a linear drive mechanism comprises a mechanical screw drive or a voice coil drive.

3. The respiratory ventilator system of claim 1, wherein the patient interface is selected from the group consisting of a mask, an intubation tube and a tracheotomy cannula.

4. The respiratory ventilator system of claim 1, wherein the pressurized air or gas source is selected from the group consisting of an air canister, a compressor, an air pump, and pressurized airline.

5. The respiratory ventilator system of claim 1, further comprising at least one of a heater and a humidifier for conditioning the air or gas.

6. A method for assisting breathing of a patient in need of same, comprising:
providing a respiratory ventilation system comprising:
an inlet configured to be connected to a pressurized air or gas source;
an outlet configured to be connected to a patient interface;
a valve in-line between the inlet and the outlet; and
a control unit configured to control the valve for controlling flow of pressurized air or gas from the source to a patient,
wherein the valve wherein the valve comprises a valve gate controlled by a linear drive servomechanism, and includes an air or gas reservoir or accumulator incorporated into a body of the valve;
connecting the respiratory ventilation system to a source of pressurized air and to a patient interface;
initiating a flow of air or gas to the respiratory ventilator system to precharge the air or gas reservoir or accumulator; and
controlling the flow of gas through the ventilation system by opening and closing the valve.

7. The method of claim 6, wherein the valve is opened and closed in response to the patient's normal breathing cycle.

8. The method of claim 6, wherein the valve is opened and closed to introduce a flow of air or gas to override the patient's normal breathing cycle.

9. The method of claim 6, wherein the patient comprises a human animal.

10. The method of claim 6, wherein the patent comprises a non-human animal.
